(19) United States
(12) Patent Application Publication
(10) Pub. No.: US 2018/0117241 A1
(43) Pub. Date: May 3, 2018

(54) SYSTEMS AND METHODS FOR IMPROVED AIR-IN-LINE DETECTION FOR INFUSION PUMPS

(71) Applicant: Smiths Medical ASD, Inc., Plymouth, MN (US)

(72) Inventors: Chad AMBORN, Minneapolis, MN (US); Timothy B. BRESINA, Shoreview, MN (US); James PARISH, Chaska, MN (US)

(73) Assignee: Smiths Medical ASD, Inc., Plymouth, MN (US)

(21) Appl. No.: 15/570,200

(22) PCT Filed: May 12, 2016
(86) PCT No.: PCT/US2016/032102

§ 371 Date: Oct. 27, 2017

Related U.S. Application Data
(60) Provisional application No. 62/161,918, filed on May 15, 2015.

(51) Int. Cl. A61M 5/14; A61M 5/36; A61M 39/28; A61M 5/142; A61M 5/168

(52) U.S. Cl. CPC A61M 5/1413; A61M 5/365; A61M 2205/12; A61M 5/14228; A61M 5/16831; A61M 39/281

(57) ABSTRACT

An administration set is configured to couple to a control module of a peristaltic infusion pump. The set can include a pressure plate and a peristaltic tube. The pressure plate can have attachment features structured to mate with corresponding attachment features of the control module such that the pressure plate is maintained in a fixed position relative to the control module. The peristaltic tube can include a first portion having first inner and outer diameters, a second portion downstream of the first portion having second inner and outer diameters, and a transition portion between the first and second portions. The peristaltic tube can be located along the pressure plate and positioned such that when the attachment features of the pressure plate and control module are mated, an expulsor of the control module engages the first portion and an air-in-line detector of the control module engages the second portion.

### CROSS-REFERENCE TO RELATED APPLICATIONS

The present application is a National Phase entry of PCT Application No. PCT/US2016/032102, filed on May 12, 2016, which claims priority to US Provisional Patent Application No. 62/161,918, filed on May 15, 2015, which are hereby fully incorporated herein by reference.

### TECHNICAL FIELD

This disclosure relates to infusion pumps, and more particularly, to systems and methods for improved air-in-line detection for infusion pumps.

### BACKGROUND

In view of the importance of avoiding introduction of gas (air or other) into the vasculature of patients, infusion pumps typically incorporate an air-in-line detector (AILD) to sense the presence of gas bubbles in lines or tubes that deliver medicaments and other infusates to patients. Many AILDs utilize ultrasonic sensing, exploiting the differing acoustic properties (e.g., transmission and reflection) of portions of a line filled with liquid, gas, or a mixture of the two. In some cases, an infusion pump can annunciate an air detection alarm when predetermined conditions are met in regard to AILD sensing, alerting a caregiver to the possibility of an air-in-line condition that may call for remediation.

Implementing a useful AILD system can require, or benefit from, careful arrangement and adjustment of hardware, and judicious choice of the predetermined conditions or parameters that define when an alarm is to be sounded. A system that is less well-implemented—that may be, for example, adversely affected by “stuck bubbles” as described in greater detail below—may fail to alarm when an air-in-line condition exists, or may provide excessive false alarms. The latter case can contribute to the annoyance of caregivers and even more problematic “alarm fatigue” (desensitization to alarms). In some cases, a poorly-implemented AILD system may be deactivated, eliminating any possible benefit. It would therefore be desirable to provide systems and methods for improved air-in-line detection for infusion pumps.

### SUMMARY

This disclosure relates to infusion pumps, and more particularly, to systems and methods for improved air-in-line detection for infusion pumps.

In an illustrative but non-limiting example, the disclosure provides an administration set configured to couple to a control module of a peristaltic infusion pump. The control module can be structured to receive the administration set along a mating side of the control module. On the mating side, the control module can include an expulsor, an upstream valve (relative to the expulsor), a downstream valve, and an air-in-line detector downstream of the downstream valve. The administration set can include a pressure plate and a peristaltic tube. The pressure plate can include a first major surface and a longitudinal axis. The pressure plate further can have attachment features structured to mate with corresponding attachment features of the control module such that the pressure plate is maintained in a fixed position relative to the control module. The peristaltic tube can include a first portion having a first inner diameter and first outer diameter, a second portion downstream of the first portion having a second inner diameter and second outer diameter, and a transition portion between the first portion and the second portion. The peristaltic tube can be located along the pressure plate along the first major surface of the pressure plate, substantially aligned with the longitudinal axis, and positioned such that when the attachment features of the pressure plate and control module are mated, the expulsor engages the first portion of the peristaltic tube and the air-in-line detector engages the second portion of the peristaltic tube.

In some cases, the downstream valve and the air-in-line detector engage the peristaltic tube at positions along the peristaltic tube separated by less than about 1.5 cm.

In some cases, the downstream valve engages the first portion of the peristaltic tube.

In some cases, the transition portion extends along the peristaltic tube for a transition length less than about 3.5 mm.

In some cases, the transition portion extends along the peristaltic tube for a transition length greater than about 2.5 mm.

In some cases, the first inner diameter is about 4.06±0.05 mm.

In some cases, the first outer diameter is about 5.79±0.05 mm.

In some cases, the second inner diameter is about 2.54±0.05 mm.

In some cases, the first outer diameter is about 4.17±0.05 mm.

In some cases, the peristaltic tube further comprises a third portion having a third inner diameter substantially the same as the first inner diameter, and a third outer diameter substantially the same as the first outer diameter.

In some cases, the first section has a first wall thickness within 10% of a second wall thickness of the second section.

In some cases, both the first second and the second section of the peristaltic tube have substantially circular cross-sections when not compressed.

In some cases, the peristaltic tube has a unitary structure.

The above summary is not intended to describe each and every example or every implementation of the disclosure. The Description that follows more particularly exemplifies various illustrative embodiments.

### BRIEF DESCRIPTION OF THE FIGURES

FIG. 1 is a schematic perspective view of an example of an infusion pump system that includes a control module and a reservoir cassette;

FIG. 2 is a schematic partial perspective view of the infusion pump system of FIG. 1 with the reservoir cassette separated from the control module;

FIG. 3 is a schematic perspective view of a remote reservoir adapter;

FIG. 4A is a schematic diagram of a standard peristaltic tube;

FIG. 4B is a schematic diagram of a high-volume peristaltic tube;

FIG. 4C is a schematic diagram of a hybrid peristaltic tube; and

FIG. 5 is a schematic perspective view of hybrid peristaltic tube with a pressure plate.

### DESCRIPTION

The following description should be read with reference to the drawings, in which like elements in different drawings may be numbered in like fashion. The drawings, which are not necessarily to scale, depict selected examples and are not intended to limit the scope of the disclosure. Although examples of construction, dimensions, and materials may be illustrated for the various elements, those skilled in the art will recognize that many of the examples provided have suitable alternatives that may be utilized.

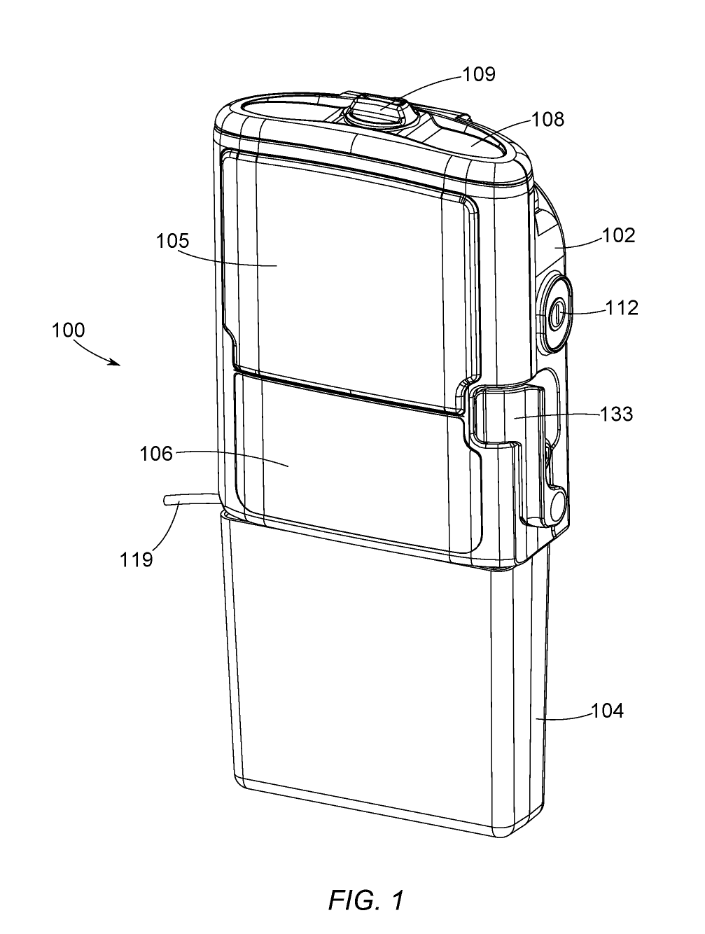

FIG. 1 is a schematic perspective view of an example embodiment of an infusion pump system 100 that includes a control module 102 and an optional reservoir cassette 104. Infusion pump system 100 can be a CADDO (Computerized Ambulatory Drug Delivery) Ambulatory Infusion Pump system from Smiths Medical ASD, Inc., although the teachings of the present disclosure are not limited to CADDO infusion pumps and may be practiced with any suitable infusion pump system.

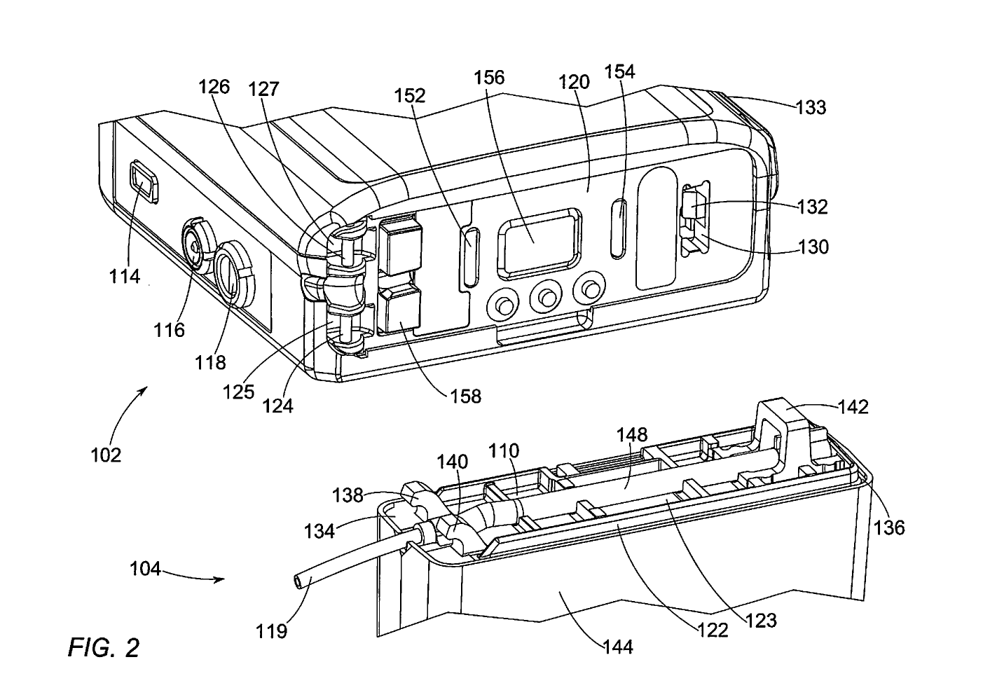

Control module 102 of infusion pump system 100 can include a user interface having a display screen 105 and a control pad 106 (buttons, etc., of the control pad are not illustrated). Control module 102 can also include a battery door 108, including a knob 109 for locking and unlocking the door 108, which can cover a battery compartment in which batteries for powering the pump system 100 can be housed. In some examples, a combination battery and wireless communication module can be present approximately where battery door 108 is illustrated. Control module 102 can also include any or all of a power switch 112, and, visible in FIG. 2 but not FIG. 1: an input/output port 114 such as a USB port or other appropriate interface for connecting pump system 100 to a computer having software designed to interface with pump system 100, a power jack 116 for connecting a power cord for powering pump 100, and a remote dose cord jack 118 for connecting a remote dose cord that provides a way to activate patient-controlled administration (or “PCA”) of doses from pump system 100.

Example infusion pump system 100 can include a replaceable reservoir cassette 104 connected to control module 102. In an embodiment, reservoir cassette 104 can house a reservoir that in turn can contain an infusate to be delivered to a patient. Tubing 119 can extend from the cassette 104 and fluidly communicates with an infusion set or catheter (not shown) to deliver the infusate to the patient. The control module 102 can be used to control the flow of infusate from cassette 104. One example of such a cassette is the CADDO Medication Cassette Reservoir from Smiths Medical ASD, Inc., though other cassettes and other hardware configurations can be used in other examples, as discussed further herein.

FIG. 2 is a schematic partial perspective view of the example of infusion pump system 100 with reservoir cassette 104 separated from control module 102, and rotated to provide views of their mating structures. Control module 102 and reservoir cassette 104 can be configured to be reversibly connected at a mating side 120 of control module 102 and pressure plate 122 of cassette 104, respectively.

In this example embodiment, mating side 120 of control module 102 can include hinge pins 124 and 126 located proximally to a first end of mating side 120, although in other examples a single hinge pin or more than two hinge pins can be employed. Hinge pins 124 and 126 are further located in hinge wells 125 and 127, respectively.

Mating side 120 of control module 102 can include a latch receptacle 130 that is located proximally to a second end of mating side 120 opposite the first end thereof. Control module 102 can include a latch mechanism 132 associated with latch receptacle 130, and a latch lever 133 to allow a user to manipulate the latch mechanism.

Pressure plate 122 of reservoir cassette 104 can include a body 123 having first 110 and second (not visible) major surfaces (top and bottom, respectively, relative to FIG. 2), a longitudinal axis and a transverse axis, first and second longitudinal sides, and first 134 and second 136 transverse ends. Pressure plate 122 can include first and second securing hooks 138, 140 extending away from first major surface 110 of body 123 proximal first transverse end 134. First and second securing hooks 138, 140 each can be structured to reversibly and hingedly couple to a corresponding one of hinge pins 124, 126. Irrespective of this example embodiment, in the present disclosure, any suitable arrangements of securing hook(s) and hinge pin(s) are contemplated.

Pressure plate 122 also can include an arch 142 extending away from first major surface 110 of body 123 proximally to second transverse end 136. Arch 142 and latch receptacle 130 of mating side 120 of control module 102 can be structured such that arch 142 is received by latch receptacle 130 as pressure plate 122 is pivoted about pins 124, 126 toward control module 102. Arch 142 can be structured to be captured by latch mechanism 132 of control module 102 and drawn toward mating side 120 of module 102 by latch mechanism 132. When arch 142 is captured by latch mechanism 132 and hooks 138, 140 are coupled to pins 124, 126, pressure plate 122 is secured thereby to control module 102.

Pressure plate 122 can be formed from any suitable material. In an embodiment, pressure plate 122 is formed from polycarbonate material, though other materials may be used. Pressure plate 122 may be joined, for example via bonding or ultrasonic welding, with a casing 144 (which may also be formed primarily of polycarbonate material) to together provide a housing of reservoir cassette 104. Reservoir cassette 104 can contain an infusate container (not shown), which may be, for example, a vinyl bag. A peristaltic tube 148 can be attached to or integrally formed with the infusate container to provide a fluid path from the infusate container to a patient, via, for example, downstream tubing 119 connected to peristaltic tube 148. In this example embodiment, peristaltic tube 148 can be substantially longitudinally located along first major surface 110 of body 123 of pressure plate 122, and can provide a fluid path that is substantially parallel to first major surface 110. In this example embodiment, the fluid path provided by peristaltic tube 148 can extend substantially to first transverse end 134 of body 123.

In this example embodiment, mating side 120 of control module 102 can include an air-in-line detector (AILD) 158 that partially surrounds peristaltic tube 148 (or another peristaltic tube, as discussed elsewhere herein) when reservoir cassette 104 (or other compatible component, as discussed elsewhere herein) is secured to control module 102. AILD 158 can include a groove into which a segment of peristaltic tube 148 resides when cassette 104 is secured to module 102. In an embodiment, AILD 158 can include at least one ultrasonic transducer located immediately adjacent to or in the groove in side 120 of module 102.

When reservoir cassette 104 is secured to control module 102, as illustrated in FIG. 1, module 102 can pump an infusate from cassette 104 through peristaltic tube 148 by way of a peristaltic-type pump mechanism. Tube engaging members visible in the example embodiment of FIG. 2 include downstream valve 152, upstream valve 154, and expulsor 156, which may engage and squeeze (compress) peristaltic tube 148 against pressure plate 122 in a coordinated manner to effect a peristaltic-type pumping action, as described for example in U.S. Pat. No. 4,559,038. In simplified brief summary, a repeatable peristaltic-type pumping cycle can include:

(I) an expulsion phase, during which upstream valve 154 is closed (e.g., valve 154 compresses peristaltic tube 148 against first major surface 110 of pressure plate 122 such that an inner lumen of tube 148 is occluded), preventing backflow toward the reservoir of cassette 104; downstream valve 152 is open (e.g., valve 152 is withdrawn away from pressure plate 122), permitting flow through a resulting open lumen of tube 148; and expulsor 156 progresses from an initial position away from pressure plate 122 to a final position that squeezes peristaltic tube 148 against pressure plate 122, such that infusate in a portion of tube 148 engaged by expulsor 156 is substantially urged or pushed downstream for delivery to the patient; and

(II) a fill phase, during which downstream valve 152 is closed, preventing backflow of infusate from a patient-side of tube 148 toward the reservoir in cassette 104; upstream valve 154 is open, permitting flow from the reservoir into tube 148; and expulsor 156 progresses from an initial position squeezing tube 148 against pressure plate 122 to a final position away from plate 122, such that as tube 148 resiliently returns from a squeezed state to an open state, fluid is drawn into tube 148 in a vicinity of expulsor 156 from the reservoir upstream in cassette 104.

Phases (I) and (II) can be repeated as needed to deliver infusate from a reservoir to a patient. Averaged over multiple cycles, increasing a rate of delivery of the infusate can be effected primarily in several ways. One way is to increase the rate of repetition of phases (I) and (II), thereby increasing the number of expulsions per time. Another way is to increase the outer diameter of the peristaltic tube, which increases the volume of fluid moved during each expulsion. Peristaltic tubes with increased outer diameters (and in some cases, increased inner diameters) are discussed further elsewhere herein.

In another example embodiment, an infusion pump can deliver fluid from a reservoir such as an IV bag that is remote, or separate from, the control module of the pump rather than from a reservoir cassette such as cassette 104. Such a configuration can be employed, for example, when a therapy calls for a volume of infusate that is greater than the capacity of an available reservoir cassette. CADD® Medication Cassette Reservoirs are commercially available, typically, in 50 ml, 100 ml, and 250 ml volume capacities. While other capacity cassettes are possible, IV bags have an ability to provide, as compared to such cassette reservoirs, very large volumes, such as 500 ml, 1000 ml, 2000 ml, and greater.

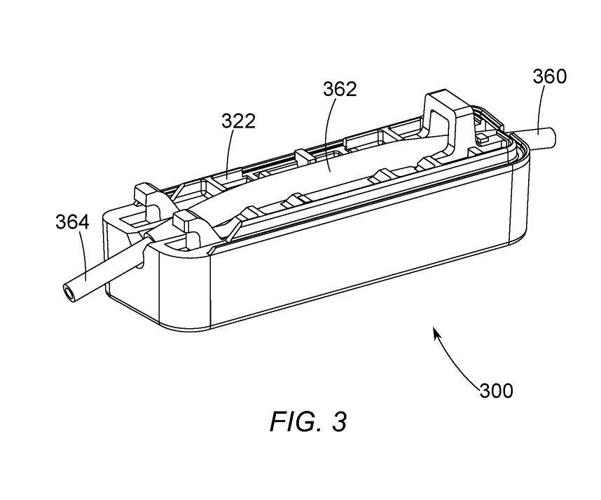

FIG. 3 is a schematic perspective view of an example of a remote reservoir adapter (“RRA”) 300 that can be employed with the aforementioned larger capacity IV bags. In this example embodiment, RRA 300 can include a pressure plate 322 that can be similar in some respects to pressure plate 122 of reservoir cassette 104. RRA 300 can include upstream tubing 360 that can extend from the IV bag (not illustrated) or other remote reservoir to pressure plate 322, where it can be fluidically coupled to a peristaltic tube 362 that extends substantially along a longitudinal axis of pressure plate 122. In this example embodiment, downstream tubing 364 can be fluidically coupled to peristaltic tube 362 opposite upstream tubing 360, and provide a fluidic path to an infusion set or catheter. Flow of infusate through such fluidically coupled segments of tubing 360, 362, 364 can be controlled by control module 102 coupled to RRA 300 in a manner similar to the coupling of control module 102 to cassette 104 as described with reference to FIG. 1. Examples of RRAs are provided as components of CADDO Administration Sets from Smiths Medical ASD, Inc.

Some therapies include relatively high rates of infusate delivery. In some cases, higher delivery rates can be practiced in connection with higher total volumes of infusate, although this is not required, and in some cases, higher delivery rates can be practiced with relatively small volumes. As stated elsewhere, one way to increase a delivery rate with a peristaltic pump mechanism such as that of FIG. 2 is to increase the outer diameter of the peristaltic tube. CADDO Administration Sets from Smiths Medical ASD, Inc. are commercially available, typically, in models including a standard or nominally-dimensioned peristaltic tube, as well as in “High Volume” models having a high-volume peristaltic tube. (While reservoir cassettes may be commercially available only with standard peristaltic tube, it is also possible, in principle, to provide a high delivery rate reservoir cassette equipped with a high-volume peristaltic tube, for applications calling for high delivery rate of a relatively small volume of infusate.)

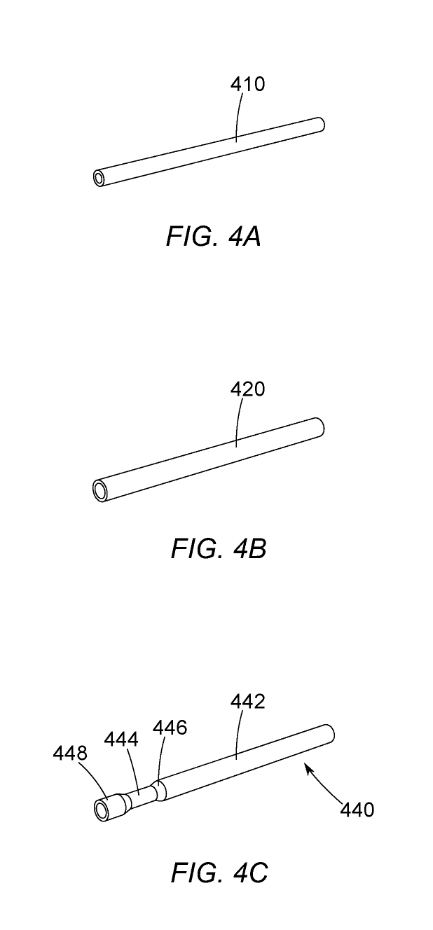

FIG. 4A is a schematic diagram of an example embodiment of a portion of standard peristaltic tube 410, and FIG. 4B is a schematic diagram of an example embodiment of a portion of high-volume peristaltic tube 420, with FIGS. 4A and 4B being provided at the same approximate scale for ease of comparing them. Standard peristaltic tube 410 can have an inner (i.e., lumen) diameter of about 2.54±0.05 mm (100±2 mil, where 1 mil=0.0254 mm) with a wall thickness of about 0.813 mm (about 32 mils), for a total outer diameter of about 4.17±0.05 mm (about 164±2 mils). A single pumping cycle on a standard peristaltic tube such as tube 410 can result in delivery of about 50 microliters of infusate. In these examples, high-volume peristaltic tube 420 can have an inner diameter of about 4.06±0.05 mm (160±2 mils) with a wall thickness of about 0.863 mm (about 34 mils), for a total outer diameter of about 5.79±0.05 mm (about 228±2 mils). A single pumping cycle on high-volume peristaltic tube such as tube 420 can result in delivery of about 100 microliters of infusate. It is to be understood that peristaltic tube 362 of FIG. 3 is illustrated as an example of a high-volume peristaltic tube.

The modular nature of infusion pump system 100 allows the same control module 102 to be paired with different cassettes and different administration sets to better accommodate specific patient therapies. For example, different reservoir volumes and different delivery rates (e.g., with different peristaltic tubes) can be selected with appropriate choices of cassettes or administration sets. Some challenges can arise for making the same control module 102 function well with such diversity of attachable hardware. The present disclosure is directed in some aspects toward improving the function of air-in-line detection in infusion pump system 100 when operated at relatively higher delivery rates as well as relatively lower delivery rates.

Referring again to FIGS. 1 and 2, AILD 158 of control module 102 can be an ultrasonic detector with an acoustic transmitter positioned on one side of the aforementioned groove that accommodates tube 148, and an acoustic receiver positioned on the other side of the groove. The presence of air in tube 148 residing in the groove can be inferred from the transmission of sound waves through the tube. The acoustic impedance of the portion of tube 148 located proximal to AILD 158 can vary depending on whether the tube contains liquid, gas, or a mixture of the two. Generally, a liquid-filled tube can transmit acoustic waves more efficiently than a tube containing gas (which can be regarded, relatively, as an acoustic open circuit). In practice, AILD 158 can be effectively “tuned” or calibrated (e.g., by adjusting threshold values used to evaluate measured quantities related to detected acoustic properties) such that gas bubbles larger than a specified threshold volume can be detected, and smaller than the threshold ignored.

Small bubbles can be ignored in some circumstances as they may present negligible risk of harm to a patient. If not ignored, their detection can result in relatively frequent annunciation of air-in-line alarms that a caregiver may, over time, learn to disregard (the so-called effect of “alarm fatigue”), potentially leading to the hazard of the caregiver disregarding a more serious alarm. Referring also to FIG. 4A, in some embodiments, AILD 158 of control module 102, when used with standard peristaltic tube 410 (2.54 mm inner diameter), can be tuned to disregard smaller bubbles having a volume of, for example, less than about 30 microliters when used with standard peristaltic tube 410 (2.54 mm inner diameter). A bubble of this volume generally can substantially bridge (extend across) the entire cross section of the tube and extend approximately 7 mm along the tube's length as described in greater detail below.

Referring also to FIG. 4B, AILD 158 can be tuned or calibrated also for use with high-volume peristaltic tube 420, as well as with standard peristaltic tube 410. It has been observed empirically, however, that some air detection problems can arise more frequently when used with high-volume peristaltic tube 420 (4.06 mm inner diameter) than when used with standard peristaltic tube 410. With relatively larger diameter high-volume peristaltic tube 420, some gaseous bubbles in the liquid that exceed a selected detection threshold (for example, the aforementioned volume of 30 microliters) can become “stuck” in the tube (remaining stationary, or otherwise moving substantially slower than the bulk flow of liquid). In comparison, in standard peristaltic tube 410, bubbles larger than the specified threshold volume for detection generally can be relied upon to progress downstream along with the pumped liquid. This characteristic of the bubbles not to become stuck in the relatively smaller standard peristaltic tube 410 can be understood from geometrical considerations. In standard peristaltic tube 410 having an inner diameter of 2.54 mm, a spherical bubble having a volume of about 8.5 microliters can substantially bridge, span, or otherwise extend across the entire cross-sectional area of the tube. Bubbles of this volume or larger that completely obstruct the tube can be pushed through the tube by pressure exerted on the upstream side of the bubble.

However, in a larger diameter tube such as high-volume peristaltic tube 420 (160 mils), the cross-section of tube 420 may not be bridged until a bubble volume reaches about 35 microliters. When a bubble does not entirely bridge the cross section of a tube, it is possible for liquid to flow around a stuck bubble without necessarily exerting enough force on the bubble to dislodge or otherwise move it. A problem can therefore arise in relatively larger diameter high-volume peristaltic tube 420 when a bubble exceeding the detection threshold is stuck in tube 420 at a location where it is detected by AILD 158. The persistence of the stuck bubble in the vicinity of AILD 158 may result in an erroneous report of presence of the same single bubble multiple times, giving an appearance of a significantly larger volume of gas in the tube. An alarm may be generated based upon the erroneously sensed (but not actual) existence of an exaggeratedly large volume of gas in tube 420. Such a false alarm may contribute to alarm fatigue, or even the deactivation of the air alarm as unreliable. It would be desirable therefore to reduce or eliminate the phenomenon of stuck bubbles in the vicinity of AILD 158.

Another aspect of operating control module 102 with both standard peristaltic tube 410 and high-volume peristaltic tube 420 is that the aforementioned groove of AILD 158 is used with both sizes of tube 410, 420. High performance air detection may be achieved more readily and consistently when AILD 158 couples to tubes of a single size or similar sizes, rather than disparate sizes.

FIG. 4C is a schematic diagram of an example embodiment of a hybrid peristaltic tube 440. In this example, hybrid peristaltic tube 440 can include a first portion 442 having a first inner diameter and a first outer diameter, a second portion 444 having a second inner diameter and a second outer diameter, and a transition portion 446 between first and second portions 442 and 444. First portion 442 can have relatively larger first inner and outer diameters as compared to inner and outer diameters of second portion 444, respectively. Hybrid peristaltic tube 440, with relatively larger diameter first portion 442 and relatively smaller diameter second portion 444, can provide improved performance for air-in-line detection (compared to high-volume peristaltic tube 420), while also providing higher infusate delivery rates (compared to standard peristaltic tube 410). In some embodiments, the second outer diameter of second portion 444 can be less than about 80%, 75%, 70%, 67%, 60%, 50%, or any other suitable percentage, of the first outer diameter of first portion 442. In some embodiments, first portion 442 can have first inner and outer diameters similar to, or the same as, the inner and outer diameters of high-volume peristaltic tube 420 (4.06 mm, and 5.79 mm, respectively). In some embodiments second portion 444 can have second inner and outer diameters similar to, or the same as, the inner and outer diameters of standard peristaltic tube 410 (2.54 mm, and 4.17 mm, respectively). In some embodiments, first portion 442 can have a first wall thickness that is within about 10% of a second wall thickness of second portion 444. In some embodiments, other dimensions can be used for first and second inner and outer diameters, provided that they function together satisfactorily for a particular embodiment of a hybrid peristaltic tube as described by example or otherwise contemplated herein.

Peristaltic tubes of the present disclosure can have substantially circular cross-sections when not compressed or otherwise influenced by external forces. Thus, in particular, any or all portions of hybrid peristaltic tube 440 can have substantially circular cross-sections when not compressed.

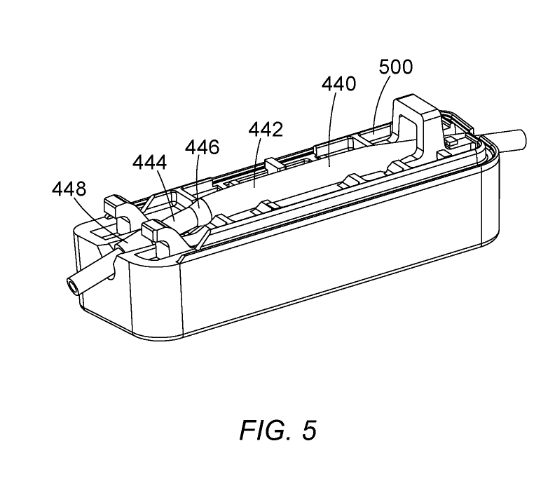

FIG. 5 is a schematic perspective view of an example of an embodiment of hybrid peristaltic tube 440 with a pressure plate 500 that is similar to pressure plates 122 and 322 in FIGS. 2 and 3. In this example, hybrid peristaltic tube 440 can be located along pressure plate 500 such that when the aforedescribed attachment features of a pressure plate such as pressure plate 500 and a control module such as control module 102 are mated, expulsor 156 of module 102 engages first portion 442 of hybrid peristaltic tube 440 and AILD 158 engages second portion 444 of tube 440. As first portion 442 can have the same or similar diameters as those of high-volume peristaltic tube 420, the volume delivered per cycle of expulsor 156 can be essentially the same for hybrid peristaltic tube 440 and high-volume peristaltic tube 420. However, with second portion 444 of hybrid peristaltic tube 440 having the same or similar diameters as those of standard peristaltic tube 410, performance of AILD 158 can be improved (as compared with performance of AILD 158 with high-volume peristaltic tube 420).

Generally, a comprehensive understanding of the dynamics of gaseous bubbles in liquid flow is beyond the scope of the present disclosure, depending on numerous factors that may include, for example, adhesive and cohesive forces, viscosity, fluid flow rate, densities, etc. Without necessarily relying upon any particular theory of fluid and/or bubble dynamics that would limit the scope of the disclosure or the claimed subject matter, it is believed that replacing a high-volume peristaltic tube such as tube 420 with a hybrid peristaltic tube such as tube 440 ameliorates the stuck bubble phenomenon and associated over-detection by an air-in-line detector such as AILD 158 in multiple ways. One way, with reference again to the examples of FIGS. 4A-C, is that second portion 444 (2.54 mm inner diameter) of hybrid peristaltic tube 440 that is engaged with AILD 158 is more readily bridged (advantageously) by bubbles than is high-volume peristaltic tube 420 or first portion 442 of the hybrid tube (4.06 mm inner diameter). Bubbles that bridge a tube generally can be moved downstream by liquid flow in the tube and generally can be less susceptible to becoming stuck and thereby causing air-in-line detection problems. Another way that use of a hybrid peristaltic tube, such as tube 440, can help remediate occurrences of stuck bubbles is that the speed of fluid in the tube increases as it progresses from the larger-diameter first portion such as portion 442, to the smaller-diameter second portion such as portion 444. This increase of fluid speed, which is a consequence of the continuity equation for fluid flow in the tube (or, perhaps somewhat colloquially, a Venturi effect or a Bernoulli effect), can increase the downstream force on bubbles that might otherwise stick or lodge in the tube proximate to the air-in-line detector under a relatively slower fluid flow.

Experimental results appear to confirm the effectiveness of prototypes of hybrid peristaltic tube 440 in reducing stuck bubbles, as compared with conventional high-volume peristaltic tubes. Video observation showed bubbles moving downstream in prototype hybrid peristaltic tubes under conditions essentially matching those that resulted in stuck bubbles in conventional high-volume peristaltic tubes. Nuisance AILD alarms relating to stuck bubbles were significantly diminished with the prototype hybrid peristaltic tubes, with control modules having AILDs tuned for standard peristaltic tube. Testing was performed with a total parenteral nutrition (TPN) infusate, a highly gaseous (i.e., tending to create gas bubbles) nutritional product that generally is delivered at a relatively high rate and at relatively high volumes, as compared with other infusates typically delivered by infusion pumps.

Any suitable technique(s) can be used to manufacture hybrid peristaltic tubes such as tube 440, including thermoforming. Thermoforming may be applied to an initial tube substantially the same or similar as high-volume peristaltic tube 420, the initial tube having uniform inner and outer diameters along its length. After thermoforming, the resulting hybrid peristaltic tube such as tube 440 can have a unitary or monolithic structure, as opposed to a structure resulting from attaching or joining multiple tubes of different diameters. Thermoforming can be used to produce hybrid peristaltic tubes such as tube 440 from conventional high-volume peristaltic tubes such as tube 420 in a manner that does not substantially change the mechanical properties of the hybrid peristaltic tube in the region of the tube engaged by an expulsor of a control module such as expulsor 156 in FIG. 2.

Referring again to FIGS. 1-5, it is therefore to be appreciated and understood that hybrid peristaltic tube 440 can be manufactured to be compatible with existing control module 102. Thermoforming or any other suitable process can be used to manufacture hybrid peristaltic tube 440 such that transition portion 446 between first portion 442 and second portion 444 is positioned (when tube 440 is located along pressure plate 500 and plate 500 is mated to control module 102) between AILD 158 and downstream valve 152. Downstream valve 152 can engage first portion 442 of tube 440, and AILD 158 can engage second portion 444 thereof. AILD 158 and downstream valve 152 can be located such that they engage the peristaltic tube (of whichever variety) at positions along the peristaltic tube separated by less than about 1.5 cm. It may be desirable to manufacture hybrid peristaltic tube 440 such that transition portion 446 extends along tube 440 for a relatively short distance and can be located away from both AILD 158 and downstream valve 152. In some embodiments, the transition portion extends along the peristaltic tube for a transition length less than about 3.5 mm. It also may be desirable that the transition between the first portion (such as portion 442) and the second portion (such as portion 444) not occur too abruptly, for example, to encourage acceptable fluid flow of the infusate. In some embodiments, the transition portion extends along the peristaltic tube for a transition length greater than about 2.5 mm.

With particular reference to FIG. 4C, hybrid peristaltic tube 440 can include a third portion 448 downstream from second portion 444, having a third inner diameter and a third outer diameter different from the second inner diameter and second outer diameter of second portion 444. As illustrated, the third inner diameter and third outer diameter of third portion 448 can be greater than the second inner diameter and second outer diameter of second portion 444. These larger third diameters can facilitate attachment of downstream tubing to hybrid peristaltic tube 440. In some embodiments, the third inner diameter of third portion 448 can be substantially the same as the first inner diameter of first portion 442, and the third outer diameter of portion 448 can be substantially the same as the first outer diameter of first portion 442.

Pressure plate 500 can be modified relative to pressure plate 322 of FIG. 3, in which peristaltic tube 362 is dimensioned like high-volume peristaltic tube 420. As discussed for example in U.S. Pat. No. 5,658,252, pressure plate 322 can include an angled surface for receiving peristaltic tube 362 that angles downward from the top major surface of pressure plate 322 as tube 362 extends away from the center of the pressure plate. For use with hybrid peristaltic tube 440, this angled surface or groove may be eliminated, or made more shallow, in the portion of the pressure plate underneath AILD 158 when the plate is coupled to control module 102. This modification can promote better engagement of hybrid peristaltic tube 440 with AILD 158.

Other hybrid peristaltic tube configurations are possible. In some embodiments (not illustrated), a hybrid peristaltic tube can include a first portion having outer and inner diameters smaller than those of standard peristaltic tube, and a second portion having outer and inner diameters the same as or similar to those of standard peristaltic tube. In such configurations, having the second portion with dimensions the same as or similar to standard peristaltic tube engaged with an AILD can result in consistent and reliable air-in-line detection performance. Having the first portion with relatively smaller outer and inner diameters engaged with an expulsor could make different performance characteristics possible, for example, possibly enabling more precise control over small volume infusate delivery.

This disclosure is to be understood to be not limited to the particular examples described herein, but rather should be understood to cover all aspects of the disclosure and equivalents thereof. Various modifications, processes, and components, as well as numerous structures to which the disclosure can be applicable, will be readily apparent to those of skill in the art upon review of the instant specification.

1. An administration set configured to couple to a control module of a peristaltic infusion pump, the control module being structured to receive the administration set along a mating side of the control module, the control module including on the mating side an expulsor, an upstream valve (relative to the expulsor), a downstream valve, and an air-in-line detector downstream of the downstream valve, the administration set comprising:
a pressure plate having a first major surface and a longitudinal axis, the pressure plate further having attachment features structured to mate with corresponding attachment features of the control module such that the pressure plate is maintained in a fixed position relative to the control module;
a peristaltic tube including a first portion having a first inner diameter and first outer diameter, a second portion downstream of the first portion having a second inner diameter and second outer diameter, and a transition portion between the first portion and the second portion, the peristaltic tube located along the pressure plate along the first major surface of the pressure plate, substantially aligned with the longitudinal axis, and positioned such that when the attachment features of the pressure plate and control module are mated, the expulsor engages the first portion of the peristaltic tube and the air-in-line detector engages the second portion of the peristaltic tube.

2. The administration set of claim 1, wherein the downstream valve and the air-in-line detector engage the peristaltic tube at positions along the peristaltic tube separated by less than about 1.5 cm.

3. The administration set of claim 1, wherein the downstream valve engages the first portion of the peristaltic tube.

4. The administration set of claim 1, wherein the transition portion extends along the peristaltic tube for a transition length less than about 3.5 mm.

5. The administration set of claim 1, wherein the transition portion extends along the peristaltic tube for a transition length greater than about 2.5 mm.

6. The administration set of claim 1, wherein the first inner diameter is about 4.06±0.05 mm.

7. The administration set of claim 1, wherein the first outer diameter is about 5.79±0.05 mm.

8. The administration set of claim 1, wherein the second inner diameter is about 2.54±0.05 mm.

9. The administration set of claim 1, wherein the first outer diameter is about 4.17±0.05 mm.

10. The administration set of claim 1, wherein the peristaltic tube further comprises a third portion having a third inner diameter substantially the same as the first inner diameter, and a third outer diameter substantially the same as the first outer diameter.

11. The administration set of claim 1, wherein the first section has a first wall thickness within 10% of a second wall thickness of the second section.

12. The administration set of claim 1, wherein both the first second and the second section of the peristaltic tube have substantially circular cross-sections when not compressed.

13. The administration set of claim 1, wherein the peristaltic tube has a unitary structure.
